(19) United States
(12) Patent Application Publication
(10) Pub. No.: US 2008/0039910 A1
(43) Pub. Date: Feb. 14, 2008

(54) Far infrared therapy unit with universal angle adjustment function

(75) Inventor: James H.C. Chen, Taipei Hsien (TW)

Correspondence Address: BIRCH STEWART KOLASCH & BIRCH, PO BOX 747, FALLS CHURCH, VA, 22040-0747, US

(73) Assignee: JOYCHIME INDUSTRIAL CORP.

(21) Appl. No.: 11/502,481

(22) Filed: Aug. 11, 2006

(51) Int. Cl. A61F 2/00

(52) U.S. Cl. 607/100

(57) ABSTRACT

A far infrared therapy unit with universal angle adjustment function includes a unit base, a universal flexible arm, which is height adjustable, and far infrared emitters. The far infrared emitters are pivotally connected to one another. One of the emitter is connected to an arched arm, which is connected to a rotation shaft. The rotational shaft is attached to the arm. The rotation shaft can rotate 360 degrees relative to the arm. The emitters can rotate 360 degrees relative to the pivotally connected emitters. Therefore, the emitters can be adjusted to any place and angle to adapt the human body. A fixing device is installed to fasten the emitters together when they are placed side by side.

### BACKGROUND OF THE INVENTION

1. Field of the Invention

The present invention relates generally to a far infrared therapy unit, and more particularly to a far infrared therapy unit with universal angle adjustment function so that the far infrared emitters are able to adapt to human body.

2. The Prior Art

Human body is a huge and delicate system. Sound, light, electricity and magnetism all can bring beneficial or harmful effects to the human body. Particularly, any natural harmful factors will affect the balance of trace elements inside the creature such that the characteristic electromagnetic wave of the creature turns into chaos and thus results in various diseases. Currently, a far infrared therapy unit widely used especially in physical therapy and beauty industries is a device for improving the above-mentioned abnormal conditions. The emitter of the far infrared therapy unit is coated with a mineral formation consisting of multiple trace elements essential to the human body. Using molecular internal motion, rotation, vibration and electron energy level excitation, the far infrared therapy unit is able to emit a special band of electromagnetic waves in the infrared range of 2 to 25 microns in wavelength. This electromagnetic wave is able to resonate with human characteristic electromagnetic wave. Therefore, the far infrared therapy unit can bring about beneficial effects to the human body, which includes rejuvenating the cells, balancing the trace elements, promoting blood circulation, releasing toxins, speeding healing and helping nutrition absorption so as to meet the therapy purposes.

One of the existing far infrared therapy units provides with an infrared emitter attached to a therapy unit base. A user has to be close to the infrared emitter to receive the far infrared radiation, which is quite inconvenient to the user. Another conventional far infrared therapy unit has an infrared emitter attached to an arm, which is installed on a vertical post. The arm can be moved up and down along the post, and rotated to a particular position. However, this type of far infrared therapy unit still cannot adapt to accommodate the human body contour.

To overcome the aforementioned disadvantages, the present invention provides an improved far infrared therapy unit with universal angle adjustment unit. The unit comprises a universal flexible arm connected to a unit base and far infrared emitters connected to one another and to a distal free end of the arm. Therefore, the far infrared therapy unit is able to adapt to human body.

### SUMMARY OF THE INVENTION

A primary objective of the present invention is to provide a far infrared therapy unit whose emitters can rotate relative to the other emitters. The emitters are pivotally connected to one another. Each emitter can rotate 360 degrees relative to the pivotally connected emitters. One of the emitters is attached to an arched arm, and the arched arm is connected to a rotation shaft. The rotation shaft is connected to a universal flexible arm. The arched arm can rotated 360 degrees relative to the universal flexible arm. Another objective of the present invention is to provide a universal flexible arm, which can be adjusted to a desired height. The arm is formed by a series of links connected with joints. Therefore, the far infrared emitters can be adjusted to any angle and arranged to any place. A further objective of the present invention is to provide a fixing device to fasten the emitters together when the emitters are placed side by side.

The present invention will be apparent to those skilled in the art by reading the following detailed description of a preferred embodiment thereof, with reference to the attached drawings, in which:

### BRIEF DESCRIPTION OF THE DRAWINGS

FIG. 1 is a perspective view of a far infrared therapy unit in accordance with the present invention;

FIG. 2 is a perspective view showing that far infrared emitters are extended;

FIG. 3 is a perspective view showing that the infrared emitters are folded into a U shape;

FIG. 4 is a schematic side view showing that a universal flexible arm is extended relative to a unit base thereof;

FIG. 5 is a detailed perspective view showing the far infrared emitters connected to the universal flexible arm; and

FIG. 6 is a detailed perspective view showing a fixing device.

### DETAILED DESCRIPTION OF PREFERRED EMBODIMENT

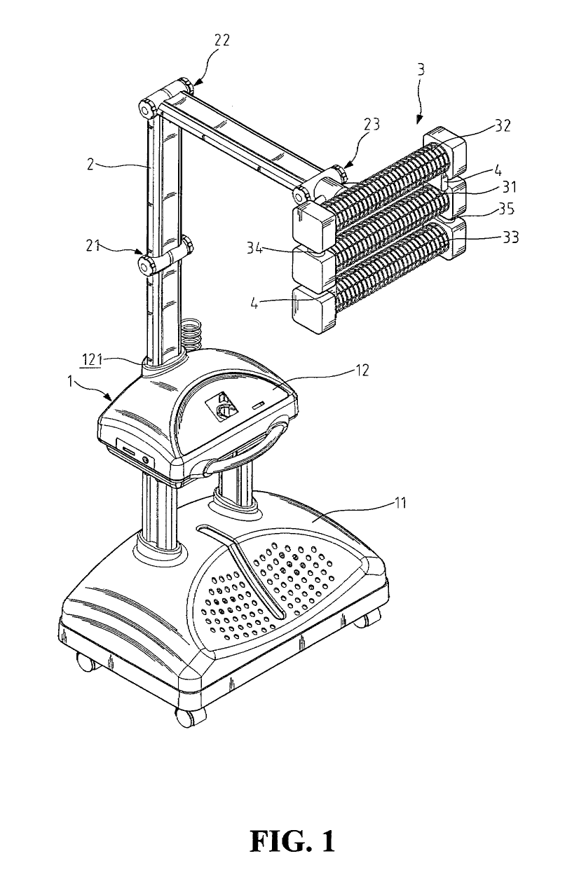

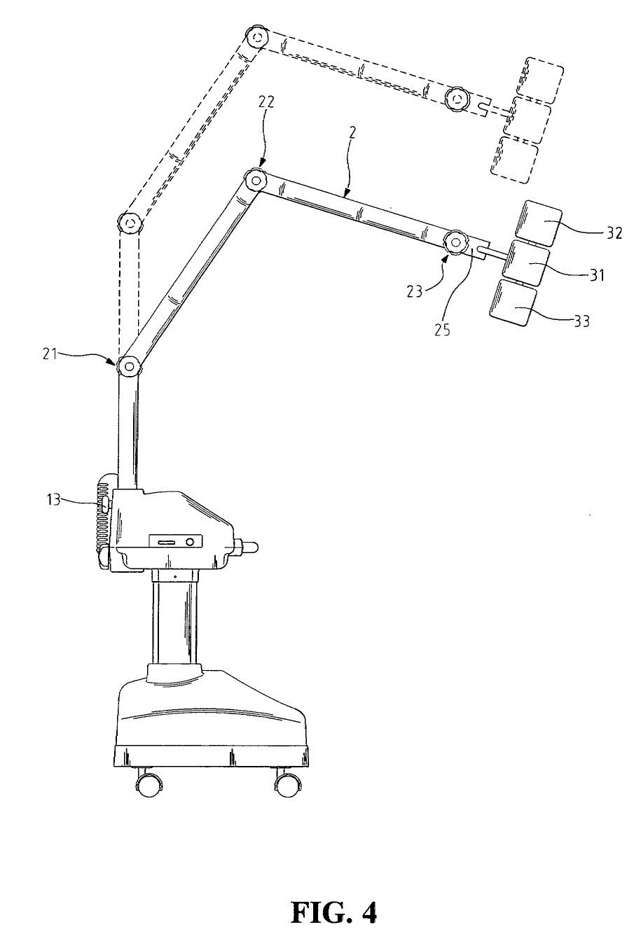

With reference to FIG. 1, a far infrared therapy unit in accordance with the present invention comprises a unit base 1, a universal flexible arm 2, an emitting device 3 and a fixing device 4. The unit base I includes a stand base 11 and a control box 12, which is mounted on columns connected to the stand base 11. The control box 12 further has a through hole 121. The universal flexible arm 2 is mounted on and passes through the through hole 121, thereby being able to move upward or downward vertically. There is a bolthole at a side surface of the control box 12 passing through to the through hole 121. When the universal flexible arm 2 is adjusted to a desired height as shown in FIG. 4, a locking bolt 13 goes through the bolthole and fastens the universal flexible arm 2 in place. The universal flexible arm 2 is a series of links connected with rotatable joints. The universal flexible arm 2 comprises a first joint 21, a second joint 22 and a third joint 23, and therefore includes multiple degrees of freedom.

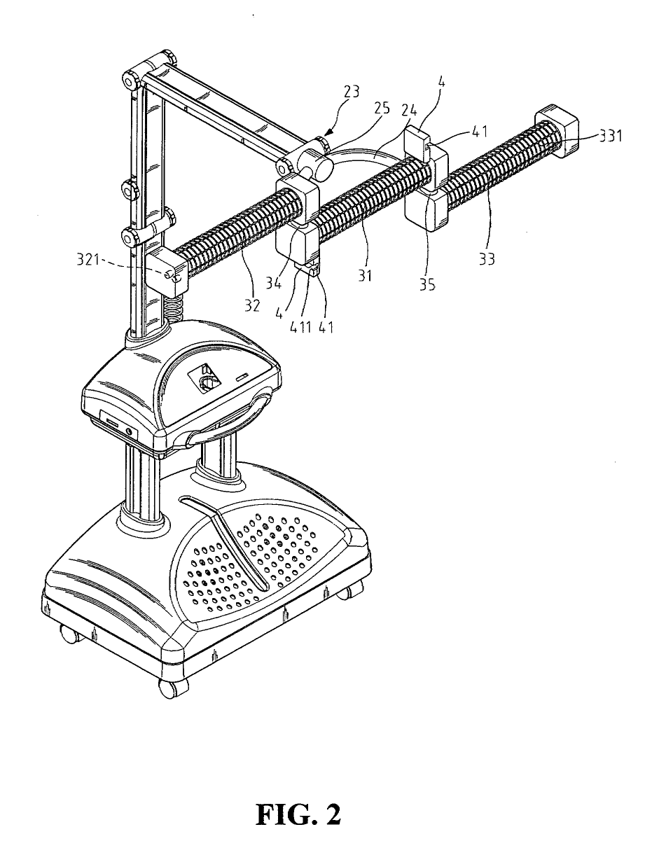

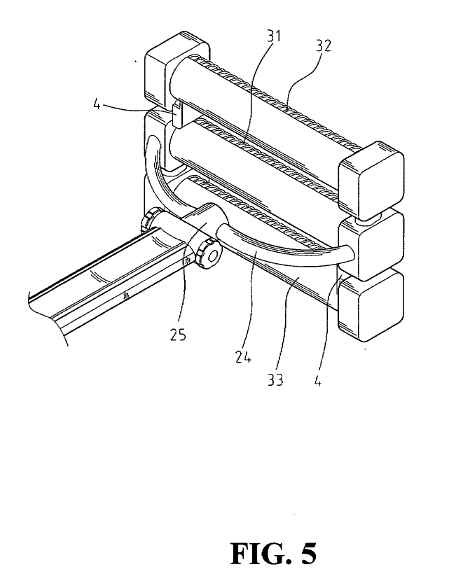

Referring to FIG. 2, a distal end of the universal flexible arm 2 provides with the third joint 23 and a rotation shaft 25. A U-shaped arched arm 24 is securely connected to the rotation shaft 25. The emitting device 3 includes a first emitter 31, a second emitter 32 and a third emitter 33. Both ends of the arched arm 24 are connected to both ends of the first emitter 31 as shown in FIGS. 2 and 5. A first pivotal rod 34 pivotally connects the first emitter 31 and the second emitter 32 together and a second pivotal rod 35 pivotally connects the first emitter 31 and the third emitter 33 together. The second emitter 32 can rotates 360 degrees relative to the first emitter 31. The third emitter 33 can rotates 360 degrees relative to the first emitter 31.

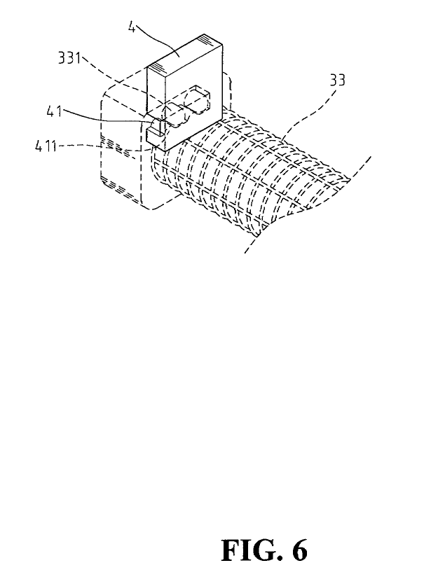

Referring to FIGS. 2, 3, 5 and 6, the fixing devices 4 are installed between the first emitter 31 and the second emitter 32, and between the first emitter 31 and the third emitter 33. The fixing devices 4 fasten the emitters together to avoid the emitters' shaking or moving when the emitters are placed side by side. The fixing devices 4 are installed at appropriate places at both ends of the first emitter 31. The fixing device 4 comprises a mortise groove 41 and at least a projection 411 at a side of the mortise groove 41 as shown in FIG. 6. The projection 411 is a small convex and do not block the entrance of the mortise groove 41. The second emitter 32 and the third emitter 33 provide with a second tenon 321 and a third tenon 331, respectively. They are installed at appropriate places at the ends of the second emitter 32 and the third emitter 33 to be fitted into the mortise grooves 41 when the emitters are retracted as shown in FIGS. 1 and 5. The diameters of the second tenon 321 and the third tenon 331 are smaller than the height of the mortise groove 41, and a little larger than the clearance between the mortise groove 41 and the projection 411. Therefore, it needs to push the emitters 32 and 33 gently to make the tenon 321 and 331 slide through the projection 411 and locked in the mortise groove 41.

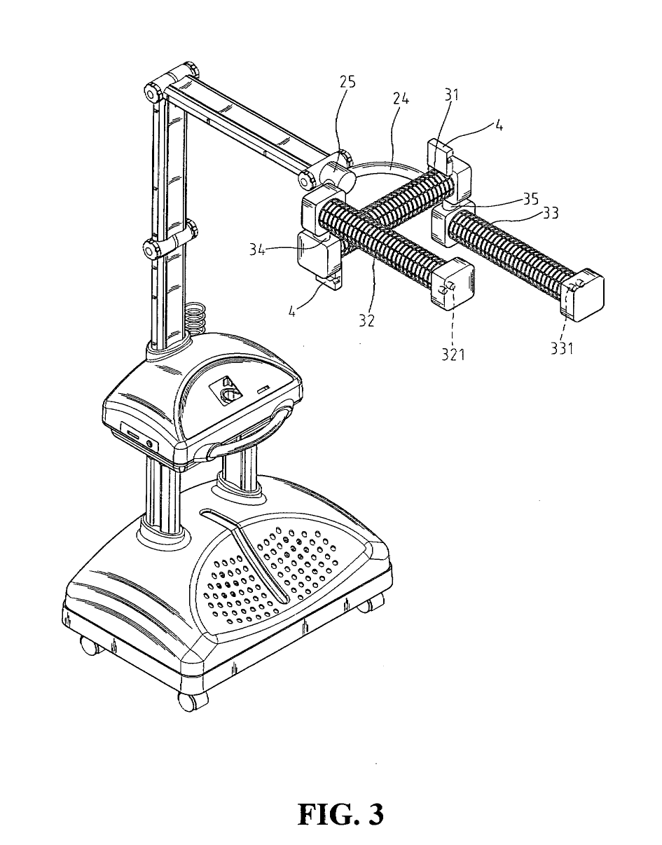

According to the present invention, in addition to the arm's height adjustment and arm links' rotation at the joints 21, 22 and 23 and rotation shaft 25, the emitters 31, 32 and 33 can also be arranged to various formations for a universal or localized treatment. The emitters 31, 32 and 33 can be retracted side by side to cover a localized area as shown in FIG. 1. The second emitter 32 and the third emitter 33 can be rotated 90 degrees relative to the first emitter 31 to form a U shape to cover a broader area as shown in FIG. 3. The second emitter 32 and the third emitter 33 can also be rotated 180 degrees relative to the first emitter 31 to form a straight line to cover an elongated area as shown in FIG. 2.

From the previous description, it is noted that the far infrared therapy unit with universal angle adjustment function of the present invention is able to adapt to different height of the user and is able to adapt to different curvatures of the user such that the user is able to conveniently use the far infrared therapy unit.

Although the present invention has been described with reference to the preferred embodiment thereof, it is apparent to those skilled in the art that a variety of modifications and changes may be made without departing from the scope of the present invention which is intended to be defined by the appended claims.

What is claimed is:

1. A far infrared therapy unit with universal angle adjustment function comprising:
a unit base;
a universal flexible arm mounted on the unit base; and
a plurality of far infrared emitters attached to the upper end of the universal flexible arm;
wherein the far infrared emitters are pivotally connected to one another;
one of the far infrared emitters is connected to an arched arm;
the arched arm is connected to a rotation shaft;
the rotation shaft is attached to the universal flexible arm;
a fixing device is installed between the pivotally connected far infrared emitters;
the fixing device provides with a mortise groove, which has a projection at a side of the mortise groove; and
the other far infrared emitter provides a tenon at an end fitted into the fixing device, whose diameter is slightly smaller than the height of the mortise groove and slightly larger than the clearance between the mortise groove and the projection.

2. The far infrared therapy unit with universal angle adjustment function as claimed in claim 1, wherein the far infrared emitters are pivotally connected to one another at one end of each emitter.

3. The far infrared therapy unit with universal angle adjustment function as claimed in claim 1, wherein the rotation shaft is capable of being rotated 360 degrees relative to the universal flexible arm.

4. The far infrared therapy unit with universal angle adjustment function as claimed in claim 2, wherein the rotation shaft is capable of being rotated 360 degrees relative to the universal flexible arm.

5. The far infrared therapy unit with universal angle adjustment function as claimed in claim 3, wherein the far infrared emitters is capable of being rotated 360 degrees relative to the pivotally connected emitters.

6. The far infrared therapy unit with universal angle adjustment function as claimed in claim 4, wherein the far infrared emitters is capable of being rotated 360 degrees relative to the pivotally connected emitters.

7. The far infrared therapy unit with universal angle adjustment function as claimed in claim 1, wherein the universal flexible arm passes through a through hole of the base unit, and is capable of moving upward or downward to a desired height.

8. The far infrared therapy unit with universal angle adjustment function as claimed in claim 1, wherein the far infrared emitters includes three emitter, a first emitter is pivotally connected to a second emitter at one end, and to a third emitter at the other end; the first emitter provides fixing devices at both ends; and the second emitter and the third emitter provide tenons at free ends.
